(19) United States
(12) Patent Application Publication
(10) Pub. No.: US 2017/0262987 A1
(43) Pub. Date: Sep. 14, 2017

(54) METHOD AND APPARATUS FOR NON-CONTACT ESTIMATION OF PULSE TRANSMIT TIME

(71) Applicant: Xerox Corporation, Norwalk, CT (US)

(72) Inventors: Nathan Gnanasambandam, Victor, NY (US); Lalit K. Mestha, Fairport, NY (US)

(21) Appl. No.: 15/065,548

(22) Filed: Mar. 9, 2016

(51) Int. Cl. G06T 7/00; A61B 5/021; A61B 5/00; G06T 7/40

(52) U.S. Cl. CPC G06T 7/0016; G06T 7/408; G06T 7/003; A61B 5/02125; A61B 5/7282; A61B 5/0077; G06T 2207/10016; G06T 2207/20032; G06T 2207/30104

(57) ABSTRACT

A method, non-transitory computer readable medium and apparatus for estimating a pulse transmit time (PTT) to calculate a blood pressure are disclosed. For example, the method includes receiving a series of video images over a period of time that includes a first location of an individual and a second location of the individual, calculating a first set of luminance values of the first location and a second set of luminance values of the second location, estimating the PTT based on an average time difference from consecutive peaks of the first set of luminance values and the second set of luminance values and transmitting the PTT to a blood pressure calculation device to calculate the blood pressure based on the PTT that is estimated.

### STATEMENT OF GOVERNMENT INTEREST

This invention was made with government support under Contract No. 1U01EB018818-01, awarded by the National Institutes of Health (NIH). The government has certain rights in this invention.

The present disclosure relates generally to estimating pulse transmit times (PTT) of an individual and, more particularly, to a method and apparatus for non-contact estimation of a PTT.

### BACKGROUND

Developing different ways of monitoring an individual's health is becoming an important issue for individuals and companies. Hardware and applications are continuously being developed to monitor various aspects of an individual's health. Blood pressure is one parameter that can provide a high level indication of whether an individual is healthy or not.

However, blood pressure may be difficult to directly measure unless using a sphygmomanometer. Pulse transmit times can be correlated to the blood pressure of an individual. Pulse transmit times are typically measured with contact sensors that are placed on an individual's finger tips. Requiring contact with a sensor can sometimes be cumbersome or dangerous for some patients (e.g., pre-mature babies in neonatal units). In addition, requiring contact may limit the ways that the pulse transmit time can be measured.

### SUMMARY

According to aspects illustrated herein, there are provided a method, non-transitory computer readable medium and apparatus for estimating a pulse transmit time (PTT) to calculate a blood pressure. One disclosed feature of the embodiments is a method that receives a series of video images over a period of time that includes a first location of an individual and a second location of the individual, calculates a first set of luminance values of the first location and a second set of luminance values of the second location, estimates the PTT based on an average time difference from consecutive peaks of the first set of luminance values and the second set of luminance values and transmits the PTT to a blood pressure calculation device to calculate the blood pressure based on the PTT that is estimated.

Another disclosed feature of the embodiments is a non-transitory computer-readable medium having stored thereon a plurality of instructions, the plurality of instructions including instructions which, when executed by a processor, cause the processor to perform operations that receive a series of video images over a period of time that includes a first location of an individual and a second location of the individual, calculate a first set of luminance values of the first location and a second set of luminance values of the second location, estimate the PTT based on an average time difference from consecutive peaks of the first set of luminance values and the second set of luminance values and transmit the PTT to a blood pressure calculation device to calculate the blood pressure based on the PTT that is estimated.

Another disclosed feature of the embodiments is an apparatus comprising a processor and a computer-readable medium storing a plurality of instructions which, when executed by the processor, cause the processor to perform operations that receive a series of video images over a period of time that includes a first location of an individual and a second location of the individual, calculate a first set of luminance values of the first location and a second set of luminance values of the second location, estimate the PTT based on an average time difference from consecutive peaks of the first set of luminance values and the second set of luminance values and transmit the PTT to a blood pressure calculation device to calculate the blood pressure based on the PTT that is estimated.

### BRIEF DESCRIPTION OF THE DRAWINGS

The teaching of the present disclosure can be readily understood by considering the following detailed description in conjunction with the accompanying drawings, in which:

FIG. 1 illustrates an example block diagram of a system of the present disclosure;

FIG. 2 illustrates an example graphical representation of the PTT calculated from video images;

FIG. 3 illustrates a flowchart of an example method for estimating a pulse transmit time (PTT) to calculate a blood pressure;

FIG. 4 illustrates a flowchart of another example method for estimating a pulse transmit time (PTT) to calculate a blood pressure; and

FIG. 5 illustrates a high-level block diagram of a computer suitable for use in performing the functions described herein.

To facilitate understanding, identical reference numerals have been used, where possible, to designate identical elements that are common to the figures.

### DETAILED DESCRIPTION

The present disclosure broadly discloses a method and apparatus for estimating a pulse transmit time (PTT) to calculate a blood pressure. As discussed above, developing different ways of monitoring an individual's health is becoming an important issue for individuals and companies. Typically, blood pressure is difficult to measure directly without a sphygmomanometer. Some methods use a PTT that is obtained via contact sensors placed on the individual. However, requiring contact with a sensor can sometimes be cumbersome or dangerous for some patients (e.g., pre-mature babies in neonatal units). In addition, requiring contact may limit the ways that the pulse transmit time can be measured.

Embodiments of the present disclosure provide a non-contact method for estimating an individual's PTT. The PTT can be used to calculate a systolic blood pressure of the individual. In one embodiment, a video image of a user is captured and various regions of interest are analyzed to estimate the individual's PTT. As a result, the methods use video of the individual that is non-invasive and does not require any equipment to be placed on the individual.

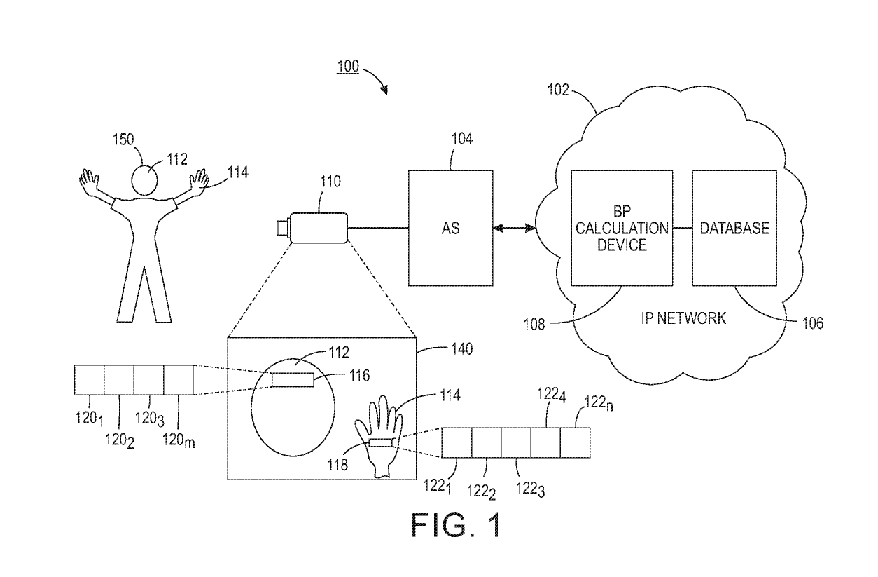

FIG. 1 illustrates an example system 100 of the present disclosure. In one embodiment, the system 100 may include a camera 110 and an application server (AS) 104. In one embodiment, the camera 110 may be a video camera that captures red, green, blue (RGB) color video (e.g., a series of video images over time) of an individual 150.

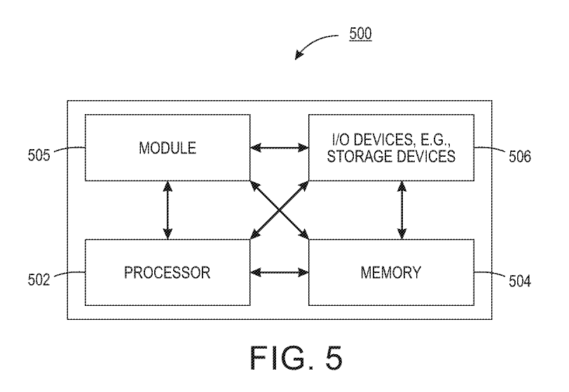

The AS 104 may be a dedicated computer or machine for performing the functions described herein. The AS 104 may include a processor and non-transitory computer readable memory that stores instructions that are executed by the processor. An example of the dedicated computer or machine is illustrated in FIG. 5 and described below.

The AS 104 may be in communication with the camera 110.

Alternatively, the video images may be captured and stored on a storage medium (e.g., a memory card, an external hard drive, and the like) and accessed by the AS 104 “offline.” The AS 104 may analyze the video images using the methods described below to estimate or calculate a PTT of the individual 150.

For example, a video image 140 may be captured of the individual 150. In one embodiment, the camera 110 may be positioned in front of the individual 150 such that the video image 140 captures at least two different locations on the individual 150. For example, the locations may include a forehead 112 of the individual 150 and a palm 114 of the individual 150.

The AS 104 may apply existing facial recognition technology to identify the forehead 112 and the palm 114 in the video image 140. In one embodiment, the AS 104 may analyze a region of interest (ROI) 116 within the area of the forehead 112 and a ROI 118 within the area of the palm 114.

As will be discussed in further detail below, the ROI 116 may be further divided into a plurality of macro pixels 120₁ to 120ₘ (herein referred to individually as a macro pixel 120 or collectively as macro pixels 120). The ROI 118 may be further divided into a plurality of macro pixels 122₁ to 122ₙ (herein referred to individually as a macro pixel 122 or collectively as macro pixels 122). It should be noted that a number of macro pixels 120 can be different, or the same, as the number of macro pixels 122.

A macro pixel 120 or 122 may be defined to be a larger grouping of pixels. For example, each macro pixel 120 or 122 may include approximately 100 pixels on which the analysis is conducted. In one embodiment, the use of the macro pixels 120 and 122 allow for a more accurate estimation of the PTT by accounting for slight variations in the skin color, movement, and the like, of the individual 150.

Luminance values may be obtained for each macro pixel 120₁ to 120ₘ and each macro pixel 122₁ to 122ₙ over a period of time. A difference in time between peaks of the luminance values in the ROI 116 of the forehead 112 of the individual 150 and peaks of the luminance values in the ROI 118 of the palm 114 of the individual 150 may be used to estimate the PTT. As a result, a series of video images 140 captured by the camera 110 may be analyze and used to estimate the PTT for the individual 150, as described in further detail below.

In one embodiment, the system 100 may be used to continuously estimate the PTT that is used to calculate the blood pressure of the individual 150. For example, while the individual 150 is watching television, the camera 110 may continuously capture the video images 140 of the individual 150. The PTT may be estimated based upon pre-determined time periods (e.g., every 10 minutes, every hour, and the like) based on the video images 140.

In one embodiment, the AS 104 may transmit the PTT to a blood pressure (BP) calculation device 108, over either a wired or wireless communication, via an Internet Protocol (IP) network 102 or the Internet. In one embodiment, the BP calculation device 108 may be in communication with a database (DB) 106 to store the PTT values received from the AS 104, as well as other information (e.g., user profiles, conversion algorithms, and the like).

It should be noted that the IP network 102 has been simplified for ease of explanation. For example, the IP network 102 may also include other network elements or access networks not shown (e.g., gateways, routers, switches, border elements, and the like).

In one embodiment, the BP calculation device 108 and the DB 106 may be located remotely from the AS 104. Alternatively, the BP calculation device 108 and the DB 106 may comprise separate functional modules within a common hardware device as the AS 104.

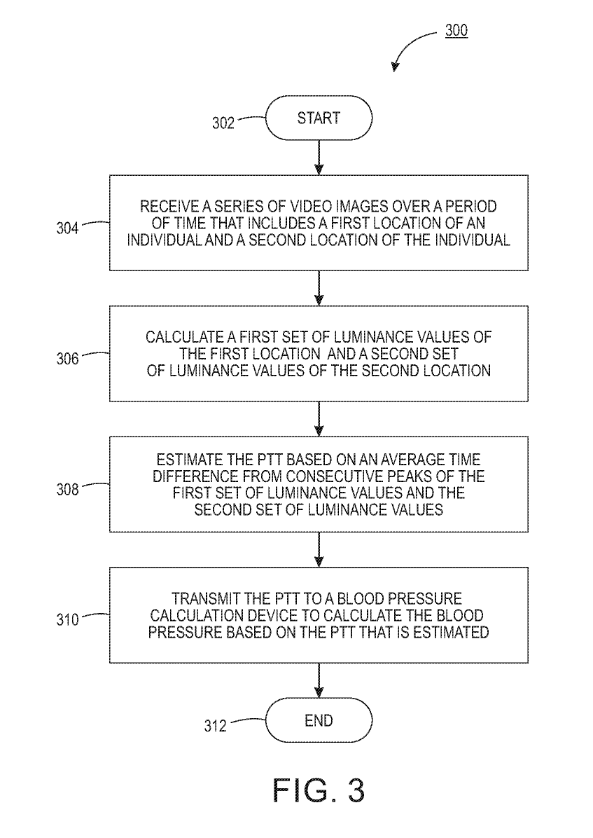

FIG. 3 illustrates a flowchart of an example method 300 for estimating a pulse transmit time (PTT) to calculate a blood pressure. In one embodiment, one or more steps or operations of the method 300 may be performed by the AS 104 or a computer as illustrated in FIG. 5 and discussed below.

At block 302, the method 300 begins. At block 304, the method 300 receives a series of video images over a period of time that includes a first location of an individual and a second location of the individual. For example, referring to FIG. 1, the first location may be the forehead 112 of the individual 150 and the second location may be the palm 114 of the individual 150.

In one embodiment, the series of video images may be an RGB color image captured by the camera 110. In one embodiment, the series of video images may be consecutive video images of a single video. In another embodiment, the series of video images may be non-consecutive video images from a plurality of videos. In other words, video images from a plurality of different videos may be stitched together for analysis.

At block 306, the method 300 calculates a first set of luminance values of the first location and a second set of luminance values of the second location. For example, for each location an ROI may be identified. The ROIs may be further divided into a plurality of macro pixels, as described in FIG. 1. The luminance values for the ROIs may be recorded over a period of time that the video images were captured.

In one embodiment, a luminance value may be calculated for each video image that is captured in the video. For example, if a video is captured by the camera 110 that includes a series of 120 video images per second (e.g., the camera 110 may capture video at 120 frames per second (fps)), then a luminance value for each macro pixel within a respective ROI of a respective location may be calculated for each one of the 120 video images.

In one embodiment, the luminance value may be the maximum luminance value of a pixel within the macro pixel. As described above, each macro pixel may comprise a plurality of pixels (e.g., approximately 100 pixels). Thus, the luminance value may be represented by the pixel with the highest luminance value. In another embodiment, the luminance value for each macro pixel within each video image may be an average of the luminance values of the pixels within the macro pixel.

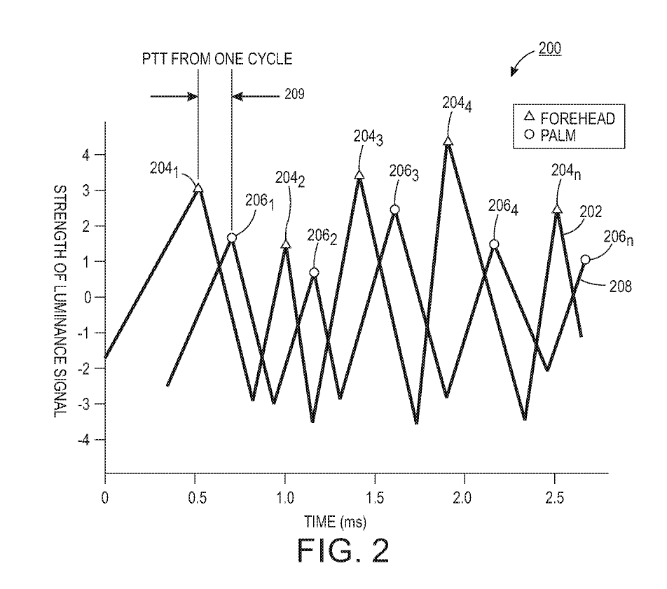

At block 308, the method 300 may estimate the PTT based on an average time difference from consecutive peaks (or peak pairs) of the first set of luminance values and the second set of luminance values. FIG. 2 illustrates a simplified diagram that illustrates the concept of time difference between consecutive peaks.

FIG. 2 illustrates an example graph 200. The graph 200 may chart time (e.g., in milliseconds (ms)) along an x-axis and strength of luminance signal along a y-axis. The graph 200 may plot the first set of luminance values 202 and the second set of luminance values 208.

However, the AS 104 may process the luminance values 202 and 208 to identify a plurality of peaks 204₁ to 204ₙ (herein also referred to individually as a peak 204 or collectively as peaks 204) associated with luminance values of the forehead 112 and a plurality of peaks 206₁ to 206ₙ (herein also referred to individually as a peak 206 or collectively as peaks 206) associated with luminance values of the palm 114. In one embodiment, luminance values 202 and 208 obtained from the video images may require further processing to identify the peaks 204 and 206 due to noise in the video images.

In one embodiment, a peak 204 or 206 may be identified based on a peak prominence threshold value and a minimum peak separation threshold value (herein also referred to generically as a first threshold and a second threshold, respectively). In one embodiment, the peak prominence threshold value may determine whether a luminance value is large enough to be considered a peak. For example, the peak prominence threshold value may be 0, 0.02, and the like; however, any value may be used. Thus, a luminance value may be considered to be a peak if the luminance value is greater than the peak prominence threshold value.

In one embodiment, the minimum peak separation threshold value may determine if there was enough time between video images to be considered to be a separate peak. In other words, the luminance value may be considered to be a peak if the peak is separated from an adjacent peak (e.g., an adjacent peak within the same set of luminance values) by an amount of time greater than the minimum peak separation threshold value (e.g., 0.5, or any other value). For example, a peak may be followed immediately by another peak that is noise (e.g., the amount of time between the two peaks is below the minimum peak separation threshold value). However, if the additional peak is considered, the peaks 204 of the forehead 112 may not align properly with the peaks 206 of the palm 114 for proper estimation of the PTT as discussed in further detail below.

After the peaks 204 and 206 are identified, the peaks 204 and 206 may be aligned. For example, the blood pumped by the heart should reach the forehead before the palm as the forehead is closer to the heart than the palm. As a result, the peaks 204 associated with luminance values of the forehead should lead (e.g., be earlier in time) the peaks 206 associated with luminance values of the palm. Thus, the identified peaks 204 and 206 may be aligned to ensure that the peaks 204 lead the peaks 206 to ensure an accurate time differential is calculated.

After the peaks 204 and 206 are identified and aligned, a time difference 209 between consecutive peaks may be calculated. In one embodiment, the term “consecutive peaks” may refer to a peak 204 from the first set of luminance values and an adjacent peak 206 from the second set of luminance values. For example, in FIG. 2 peak 204₁ and the adjacent peak 206₁ may be referred to as consecutive peaks. Similarly, peak 204₂ and the adjacent peak 206₂ may also be referred to as consecutive peaks. In other words, the term “consecutive peaks” is not referring to consecutive peaks within the same set of luminance values (e.g., peaks 204₃ and peaks 204₄ are not considered “consecutive peaks”).

In one embodiment, the time difference 209 between consecutive peaks may be an estimated PTT from one cycle of the heartbeat. The estimated PTT may be calculated based on an average time difference of all the time differences 209 between consecutive peaks of the peaks 204 and 206.

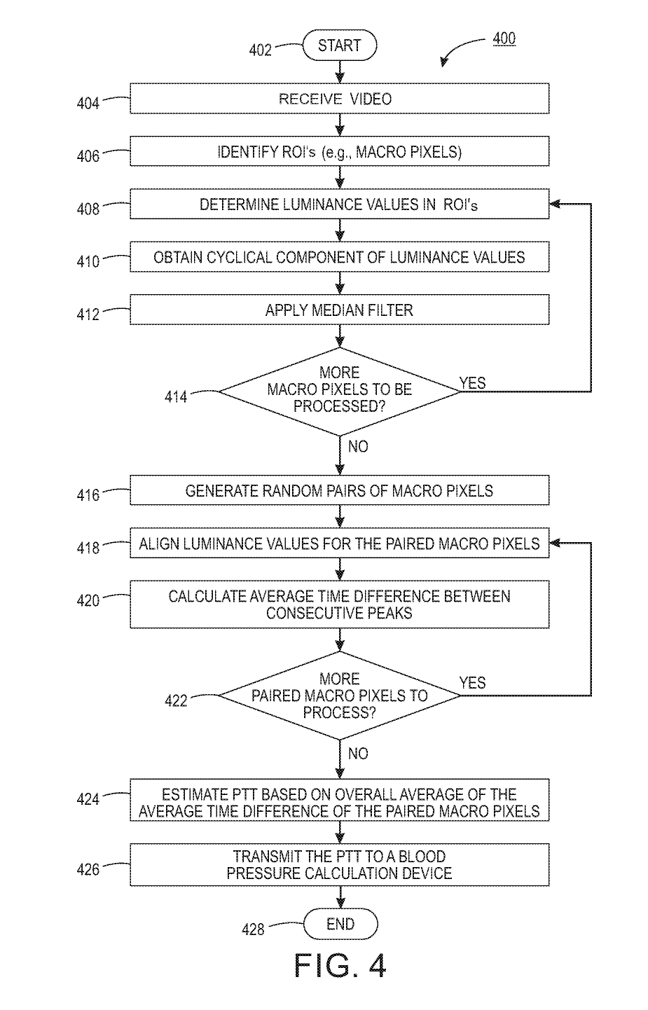

As will be discussed in further detail below with reference to FIG. 4, block 306 may be repeated for each one of the macro pixels 120 and 122. In addition, block 308 may be repeated for each randomly paired set of macro pixels 120 and 122 to obtain an overall average time difference that may represent the estimated PTT.

At block 310, the method 300 transmits the PTT to a blood pressure calculation device to calculate the blood pressure based on the PTT that is estimated. For example, the PTT may be used to calculate a systolic blood pressure value of the individual 150 based on the height, weight, gender, and the like of the individual 150. Said another way, the AS 104 may generate and output the estimated PTT that can be used by the blood pressure calculation device to calculate the blood pressure of the individual 150. At block 312, the method 300 ends.

FIG. 4 illustrates a more detailed flowchart of an example method 400 for estimating a pulse transmit time (PTT) to calculate a blood pressure. In one embodiment, one or more steps or operations of the method 400 may be performed by the AS 104 or a computer as illustrated in FIG. 5 and discussed below.

At block 402, the method 400 begins. At block 404, the method 400 receives a video. For example, the video may be an RGB video that includes a series of images recorded over a period of time of an individual. The video may include at least two different locations on the individual (e.g., a forehead and a palm of the individual).

At block 406, the method 400 identifies an ROI of the forehead and an ROI of the palm. The ROIs may be further divided into a plurality of macro pixels.

At block 408, the method may determine luminance values in the ROIs. For example, the luminance values may be determined for each macro pixel in the ROI of the forehead and each macro pixel in the ROI of the palm. In one embodiment, the luminance value may be a maximum luminance value associated with a pixel within a respective macro pixel. In another embodiment, the luminance value may be an average of the luminance value of each pixel within the respective macro pixel.

In one embodiment, each video image of the series of video images of the video that is captured may have a plurality of luminance values associated with it. For example, a luminance value may be determined for each macro pixel of the ROI of the forehead and a luminance value may be determined for each macro pixel of the ROI of the palm.

At block 408, the method 400 may obtain a cyclical component of luminance values. For example, the luminance values (yₜ) may be comprised of a trend component (τₜ), a cyclical component (CO and an error component (εₜ) according to the relationship shown in Equation 1 below:

yₜ=τₜ+Cₜ+εₜ Equation 1:

Equation 1 may be rearranged to solve for Cₜ+εₜ using an optimization function. One example of an optimization function is the Hodrick and Prescott Decomposition described below in Equation 2.

$${min}_{τ} {( {\sum }_{t = 1}^{T} {( {y}_{t} - {τ}_{t} )}^{2} + λ {\sum }_{t = 2}^{T - 1} [ {τ}_{t + 1} - {τ}_{t} ) - ( {τ}_{t} - {τ}_{t - 1} ) ]}^{2} ) , \qquad Equation 2$$

where t is time from 1 to T and λ is a scalar value that is suitably chosen. For example, a high value of λ makes the luminance values a straight line. In one embodiment, λ may be chosen to be very large (e.g., λ=10⁶).

At block 412, the method 400 may apply a median filter. The median filter may be applied to smooth out the variations and outliers caused by noise or movement of the individual in the luminance values that are determined from each macro block.

In one embodiment, the median filter may apply a sliding window using a pre-defined number of data points to calculate the median of the data points within the sliding window. In one embodiment, the pre-defined number may be an odd number (e.g., 51). To illustrate, the first 6 data points may be 3, 4, 7, 5, 9 and 6. Using a sliding window that has a pre-defined size of 5 data points, the sliding window may first calculate the median of the data points 3, 4, 7, 5 and 9 to be 5. Then, the sliding window may calculate the median of the next 5 data points 4, 7, 5, 9 and 6 as 6, and so forth.

At block 414, the method 400 may determine if more macro pixels need to be processed. If all of the macro pixels in each ROI identified in block 406 have not been processed, then the method 400 may return to block 408 and the blocks 408-414 may be repeated for each additional macro pixel. However, if all of the macro pixels have been processed, the method 400 may proceed to block 416.

At block 416, the method 400 generates random pairs of macro pixels. For example, referring back to FIG. 1, each macro pixel 120 may be paired with a macro pixel 122. In other words, each macro pixel 120 of the ROI 116 of the forehead 112 may be randomly paired with a macro pixel 122 of the ROI 118 of the palm 114. Since the number of macro pixels 120 may not be equal to the number of macro pixels 122, some macro pixels 120 or 122 may be randomly paired multiple times.

At block 418, the method 400 may align the luminance values for the paired macro pixels. In one embodiment, the luminance values at block 418 may refer to the cyclical portions of the luminance values that were calculated in block 410. As discussed above in FIG. 3, first the peaks for the luminance values are identified. For example, a peak prominence threshold value and a minimum peak separation threshold value may be applied to the luminance values to identify the peaks.

In addition, the peaks of the luminance values of a macro pixel of the forehead are aligned with the peaks of the luminance values of a macro pixel of the palm that are randomly paired. As discussed above, the peaks of the macro pixel of the forehead should lead the peaks of the macro pixel of the palm. In addition, each peak of the macro pixel of the forehead should be paired with a peak of the macro pixel of the palm (e.g., peak pairs). This is because each heartbeat should have a luminance signal detected in the forehead and a corresponding luminance signal detected in the palm.

Error correction may be applied to the luminance values to remove any peaks that do not meet the peak prominence threshold value and minimum peak separation threshold and to remove any outlier peaks that are not paired with a corresponding peak. For example, a peak of the macro pixel of the forehead should be paired with only a single corresponding, adjacent or consecutive peak from the macro pixel of the palm. In addition, the error correction may remove any peaks from a macro pixel of the palm that lead a corresponding peak from a macro pixel of the forehead.

At block 420, the method 400 calculates an average time difference between consecutive peaks. Referring to FIG. 2, the time difference 209 between consecutive peaks 204 and 206 for one cycle may be calculated for all of the peaks 204 and 206. Then an average of the time differences 209 may be calculated.

At block 422, the method 400 may determine if more paired macro pixels need to be processed. If additional paired macro pixels need to be processed, the method 400 may return to block 418 and repeat blocks 418 to 422. However, if all of the paired macro pixels have been processed, the method 400 may proceed to block 424.

At block 424, the method 400 estimates the PTT based on an overall average of the average time difference of the paired macro pixels. For example, each pair of macro pixels may have an average time difference. The average time difference for each paired macro pixel may then be averaged again to calculate the overall average time difference. The overall average time difference may represent the estimated PTT for the individual.

At block 426, the method 400 may transmit the PTT to a blood pressure calculation device. As noted above, the PTT may be used to calculate a systolic blood pressure of the individual. At block 428, the method 400 ends.

It should be noted that although not explicitly specified, one or more steps, functions, or operations of the methods 300 and 400 described above may include a storing, displaying and/or outputting step as required for a particular application. In other words, any data, records, fields, and/or intermediate results discussed in the methods can be stored, displayed, and/or outputted to another device as required for a particular application. Furthermore, steps, functions, or operations in FIGS. 3 and 4 that recite a determining operation, or involve a decision, do not necessarily require that both branches of the determining operation be practiced. In other words, one of the branches of the determining operation can be deemed as an optional step.

FIG. 5 depicts a high-level block diagram of a computer that can be transformed to into a machine that is dedicated to perform the functions described herein. As a result, the embodiments of the present disclosure improve the operation and functioning of the computer to improve non-contact methods for estimating a pulse transmit time (PTT) to calculate a blood pressure, as disclosed herein.

As depicted in FIG. 5, the computer 500 comprises one or more hardware processor elements 502 (e.g., a central processing unit (CPU), a microprocessor, or a multi-core processor), a memory 504, e.g., random access memory (RAM) and/or read only memory (ROM), a module 505 for estimating a pulse transmit time (PTT) to calculate a blood pressure, and various input/output devices 506 (e.g., storage devices, including but not limited to, a tape drive, a floppy drive, a hard disk drive or a compact disk drive, a receiver, a transmitter, a speaker, a display, a speech synthesizer, an output port, an input port and a user input device (such as a keyboard, a keypad, a mouse, a microphone and the like)). Although only one processor element is shown, it should be noted that the computer may employ a plurality of processor elements. Furthermore, although only one computer is shown in the figure, if the method(s) as discussed above is implemented in a distributed or parallel manner for a particular illustrative example, i.e., the steps of the above method(s) or the entire method(s) are implemented across multiple or parallel computers, then the computer of this figure is intended to represent each of those multiple computers. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computer or any other hardware equivalents, e.g., computer readable instructions pertaining to the method(s) discussed above can be used to configure a hardware processor to perform the steps, functions and/or operations of the above disclosed methods. In one embodiment, instructions and data for the present module or process 505 for estimating a pulse transmit time (PTT) to calculate a blood pressure (e.g., a software program comprising computer-executable instructions) can be loaded into memory 504 and executed by hardware processor element 502 to implement the steps, functions or operations as discussed above in connection with the exemplary methods 300 and 400. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 505 for estimating a pulse transmit time (PTT) to calculate a blood pressure (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. More specifically, the computer-readable storage device may comprise any physical devices that provide the ability to store information such as data and/or instructions to be accessed by a processor or a computing device such as a computer or an application server.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A method for estimating a pulse transmit time (PTT) to calculate a blood pressure, comprising:
receiving, by a processor, a series of video images over a period of time that includes a first location of an individual and a second location of the individual;
calculating, by the processor, a first set of luminance values of the first location and a second set of luminance values of the second location;
estimating, by the processor, the PTT based on an average time difference from consecutive peaks of the first set of luminance values and the second set of luminance values; and
transmitting, by the processor, the PTT to a blood pressure calculation device to calculate the blood pressure based on the PTT that is estimated.

2. The method of claim 1, wherein the first location comprises a palm of the individual.

3. The method of claim 1, wherein the second location comprises a forehead of the individual.

4. The method of claim 1, wherein the series of video images comprise consecutive video images of a single video.

5. The method of claim 1, wherein the series of video images comprise non-consecutive video images from a plurality of videos.

6. The method of claim 1, wherein the calculating further comprises:
recording, by the processor, the first set of luminance values of a region of interest (ROI) of the first location over the period of time;
recording, by the processor, the second set of luminance values of a ROI of the second location over the period of time; and
identifying, by the processor, each peak in the first set of luminance values of the ROI of the first location and each peak in the second set of luminance values of the ROI of the second location.

7. The method of claim 6, wherein the first set of luminance values of the ROI of the first location and the second set of luminance values of the ROI of the second location comprise a cyclical component.

8. The method of claim 6, wherein the ROI of the first location comprises a first plurality of macro pixels and the ROI of the second location comprises a second plurality of macro pixels.

9. The method of claim 8, wherein the first set of luminance values of each macro pixel of the first plurality of macro pixels and the second set of luminance values of each macro pixel of the second plurality of macro pixels comprise a maximum luminance value of a pixel within a respective macro pixel.

10. The method of claim 9, further comprising:
applying, by the processor, an optimization function to the first set of luminance values of the each macro pixel of the first plurality of macro pixels and the second set of luminance values of the each macro pixel of the second plurality of macro pixels; and
applying, by the processor, a median filter of a pre-defined size to the first set of luminance values of the each macro pixel of the first plurality of macro pixels and the second set of luminance values of the each macro pixel of the second plurality of macro pixels after the optimization function is applied.

11. The method of claim 10, wherein the comparing is performed between a randomly selected pair of macro pixels comprising a first macro pixel of the first plurality of macro pixels and a second macro pixel of the second plurality of macro pixels.

12. The method of claim 11, wherein the PTT that is estimated is an average value of an average time difference from the consecutive peaks for each randomly selected pair of macro pixels of a plurality of randomly selected pair of macro pixels.

13. The method of claim 6, wherein a peak is identified when a luminance value is greater than a first threshold and is separated from an adjacent peak by an amount of time greater than a second threshold.

14. The method of claim 6, wherein the estimating further comprises:
aligning, by the processor, the first set of luminance values with the second set of luminance values such that the each peak in the first set of luminance values of the ROI of the first location leads the each peak in the second set of luminance values of the ROI of the second location.

15. A non-transitory computer-readable medium storing a plurality of instructions, which when executed by a processor, cause the processor to perform operations for estimating a pulse transmit time (PTT) to calculate a blood pressure, the operations comprising:
receiving a series of video images over a period of time that includes a first location of an individual and a second location of the individual;
calculating a first set of luminance values of the first location and a second set of luminance values of the second location;
estimating the PTT based on an average time difference from consecutive peaks of the first set of luminance values and the second set of luminance values; and
transmitting the PTT to a blood pressure calculation device to calculate the blood pressure based on the PTT that is estimated.

16. The non-transitory computer-readable medium of claim 15, wherein the calculating further comprises:
recording the first set of luminance values of a region of interest (ROI) of the first location over the period of time;
recording the second set of luminance values of a ROI of the second location over the period of time; and
identifying each peak in the first set of luminance values of the ROI of the first location and each peak in the second set of luminance values of the ROI of the second location.

17. The non-transitory computer-readable medium of claim 16, wherein the ROI of the first location comprises a first plurality of macro pixels and the ROI of the second location comprises a second plurality of macro pixels.

18. The non-transitory computer-readable medium of claim 17, wherein the first set of luminance values of each macro pixel of the first plurality of macro pixels and the second set of luminance values of each macro pixel of the second plurality of macro pixels comprise a maximum luminance value of a pixel within a respective macro pixel.

19. The non-transitory computer-readable medium of claim 18, further comprising:
applying an optimization function to the first set of luminance values of the each macro pixel of the first plurality of macro pixels and the second set of luminance values of the each macro pixel of the second plurality of macro pixels; and
applying a median filter of a pre-defined size to the first set of luminance values of the each macro pixel of the first plurality of macro pixels and the second set of luminance values of the each macro pixel of the second plurality of macro pixels after the optimization function is applied.

20. A method for estimating a pulse transmit time (PTT) to calculate a blood pressure, the method comprising:
receiving, by a processor, a first video over a period of time of a forehead of an individual and a second video of a palm of the individual;
identifying, by the processor, a forehead region of interest (ROI) comprising a first plurality of macro pixels in the first video of the forehead and a palm ROI comprising a second plurality of macro pixels in the second video of the palm;
determining, by the processor, forehead luminance values for each one of the first plurality of macro pixels over the time period and palm luminance values for each one of the second plurality of macro pixels over the period of time;
identifying, by the processor, peaks in the forehead luminance values and the palm luminance values;
pairing, by the processor, a macro pixel of the first plurality of macro pixels with a macro pixel of the second plurality of macro pixels to create a plurality of macro pixel pairs;
aligning, by the processor, the forehead luminance values with the palm luminance values of each one of the plurality of macro pixel pairs;
calculating, by the processor, for each one of the plurality of macro pixel pairs an average time difference between consecutive peaks, wherein the consecutive peaks comprise a peak of the forehead luminance values and an adjacent peak of the palm luminance values;
estimating, by the processor, the PTT based on an overall average of the average time difference calculated for each one of the plurality of macro pixel pairs; and
transmitting, by the processor, the PTT to a blood pressure calculation device to calculate the blood pressure based on the PTT that is estimated.
